(19) United States
(12) Patent Application Publication
(10) Pub. No.: US 2006/0286040 A1
(43) Pub. Date: Dec. 21, 2006

(54) Medicament compositions comprising a heterocyclic compound and an anticholinergic

(75) Inventors: Christopher J. Montague Meade, Maselheim (DE); Michel Pairet, Biberach (DE); Michael P. Pieper, Biberach (DE); Yuzuru Abe, Sunto-gun (JP); Ichiro Miki, Sunto-gun (JP)

Correspondence Address: DARBY & DARBY P.C., P. O. BOX 5257, NEW YORK, NY, 10150-5257, US

(73) Assignee: Kyowa Hakko Kogyo Co., Ltd., Tokyo (JP)

(21) Appl. No.: 10/567,127

(22) PCT Filed: Aug. 5, 2004
(86) PCT No.: PCT/JP04/11578

§ 371 Date: Mar. 6, 2006

(30) Foreign Application Priority Data
Aug. 6, 2003 (EP) 03017943.6

(51) Int. Cl. A61K 9/14; A61K 31/4747; A61K 31/4745; A61L 9/04

(52) U.S. Cl. 424/46; 514/278; 514/291

(57) ABSTRACT

The present invention relates to novel pharmaceutical compositions based on anticholinergics of the formula 1; wherein X⁻, R¹ ? and Ar are defined as in claim 1, and a heterocyclic compound of the formula 2. Furthermore, the invention relates to processes for preparing them and their use in the treatment of respiratory complaints.

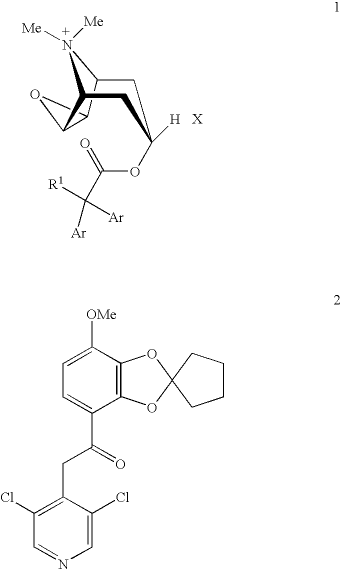

### TECHNICAL FIELD

The present invention relates to novel pharmaceutical compositions based on anticholinergics such as an anticholinergic of the formula 1 and a heterocyclic compound of the formula 2, both as defined hereinafter, and to the use of such a composition. Furthermore the present invention relates to capsules containing an inhalable powder and to the use of such capsules. In addition, this invention relates to the use of an inhalable solution. The present invention also relates to a method of prophylaxis of, treating of, or reducing the exacerbations associated with, a pulmonary disease in a patient in need thereof. Furthermore the invention relates to a package comprising a pharmaceutical composition and to an inhaler.

### BACKGROUND ART

The compound of the formula 2 and its manufacture are known from WO 96/36624. The compounds described therein exhibit phosphodiesterase (PDE) IV inhibitory activity.

A combination of PDE IV inhibitors and tiotropium for treating obstructive airways and other inflammatory diseases is described in the WO 02/096423.

Furthermore, a method of treating pulmonary diseases such as obstructive disease or asthma by administering a PDE IV inhibitor in combination with an anticholinergic agent is described in the WO 03/011274.

### SUMMARY OF THE INVENTION

The present invention relates to novel pharmaceutical compositions based on anticholinergics such as an anticholinergic of the formula 1 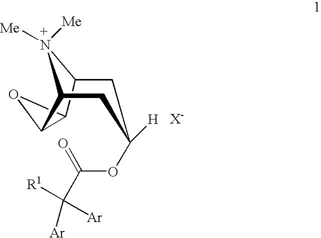
wherein
X⁻ represents chlorine, bromine, iodine, methanesulphonate or trifluoromethanesulphonate;
R¹ represents hydroxy or methyl;
Ar represents phenyl or thienyl, preferably 2-thienyl;
   and a heterocyclic compound of the formula 2 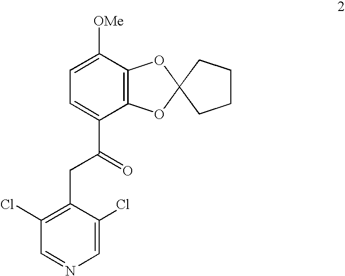
   optionally in the form of a pharmacologically acceptable acid addition salt thereof, optionally in the form of a solvate or hydrate and optionally together with a pharmaceutically acceptable excipient.

The present invention further relates to capsules characterised in that they contain an inhalable powder which contains the anticholinergics such as an anticholinergic of the formula 1 and the compound of the formula 2 in admixture with suitable physiologically acceptable excipients selected from among the monosaccharides, disaccharides, oligo- and polysaccharides, polyalcohols, salts, or mixtures of these excipients with one another.

This invention also relates to the use of a capsule according to this invention in an inhaler.

Furthermore, this invention relates to the use of a pharmaceutical composition in the form of an inhalable solution according to this invention for nebulising in an inhaler, preferably an inhaler according to WO 91/14468 or an inhaler as described according to the FIGS. 6a and 6b of WO 97/12687.

In addition, this invention relates to the use of a pharmaceutical composition according to this invention for preparing a medicament for the prophylaxis of, treating of, or reducing the exacerbations associated with pulmonary diseases, in particular of inflammatory or obstructive diseases of the respiratory tract.

This invention also relates to the prophylaxis of, treating of, or reducing the exacerbations associated with pulmonary diseases, in particular inflammatory or obstructive diseases of the respiratory tract, by administering to a patient in need thereof an effective amount of a pharmaceutical composition according to this invention either in a single combined form, separately, or separately and sequentially where the sequential administration is close in time, or remote in time.

Furthermore, this invention relates to a package comprising a pharmaceutical composition according to this invention for insertion into a device of simultaneous or sequential delivery of said pharmaceutical composition in the form of an aerosol or dry powder dispersion, to a mammal in need of treatment.

The present invention also relates to an inhaler comprising a pharmaceutical composition according to the invention for simultaneous or sequential delivery of said pharmaceutical composition in the form of an aerosol or dry powder dispersion, to a mammal in need of treatment.

### DETAILED DESCRIPTION OF THE INVENTION

Surprisingly, an unexpectedly beneficial therapeutic effect can be observed in the treatment of inflammatory and/or obstructive diseases of the respiratory tract if an anticholinergic such as an anticholinergic of the formula 1 is used with a compound of the formula 2. Among other beneficial effects, a synergistic effect can be observed. A synergistic effect may have the advantage that the pharmaceutical combinations according to the invention can be used in smaller doses than would be the case with the individual compounds used in monotherapy in the usual way.

The pharmaceutical composition of the present invention is useful in the treatment and/or prophylaxis of obstructive airways diseases, especially of asthma, chronic obstructive pulmonary disease (COPD) and other obstructive airways diseases exacerbated by heightened bronchial reflexes, inflammation, bronchial hyper-reactivity and bronchospasm, in particular COPD.

In particular, the combination of an anticholinergic of the formula 1 and a compound of the formula 2 are useful in the treatment of respiratory diseases and conditions comprising: asthma, acute respiratory distress syndrome, chronic pulmonary inflammatory disease, bronchitis, chronic bronchitis, chronic obstructive pulmonary (airway) disease, including the pulmonary symptoms associated with cystic fibrosis and mucoviscidosis; and silicosis; or immune diseases and conditions comprising: allergic rhinitis and chronic sinusits.

The effects mentioned above may be observed both when the two active substances are administered simultaneously in a single active substance formulation and when they are administered successively in separate formulations. According to the invention, it is preferable to administer the two active substance ingredients simultaneously in a single formulation.

Accordingly, in one aspect, the present invention relates to a pharmaceutical composition, characterised in that it contains an anticholinergic of the formula 1 in combination with a compound of the formula 2. The compound of the formula 2 may be present in the form of the pharmacologically acceptable acid addition salts thereof.

Any reference to the above compound of the formula 2 includes within the scope of the present invention a reference to any pharmacologically acceptable acid addition salts thereof which may exist. By the physiologically acceptable acid addition salts which may be formed from a compound of the formula 2 are meant, for example, pharmaceutically acceptable salts selected from the salts of hydrochloric acid, hydrobromic acid, sulphuric acid, phosphoric acid, methanesulphonic acid, acetic acid, fumaric acid, succinic acid, lactic acid, citric acid, tartaric acid or maleic acid. Preferred salts of the compound of the formula 2 according to the invention are those selected from the salts of hydrochloric acid, hydrobromic acid, sulphuric acid, phosphoric acid, methanesulphonic acid and acetic acid.

In the abovementioned salts of the anticholinergic of the formula 1 the corresponding cation represents the pharmacologically active ingredient. Within the scope of the present patent application any reference to the above cations is indicated by the use of the term 1′. Any reference to anticholinergics of the formula 1 naturally also includes a reference to the components 1′.

By the salts of the anticholinergic of the formula 1 which may be used within the scope of the present invention are meant the compounds which contain, in addition to the corresponding cation as counter-ion (anion), chloride, bromide, iodide, methanesulphonate or para-toluenesulphonate. Within the scope of the present invention, the methanesulphonate, chloride, bromide and iodide are preferred of all the salts of the anticholinergic of the formula 1, the methanesulphonate and bromide being of particular importance. The most preferred meaning of X⁻ is a bromide anion.

According to a first preferred embodiment of this invention, the pharmaceutical composition comprises a compound of the formula 1a 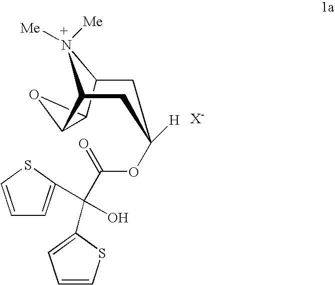
wherein
X⁻ represents chlorine, bromine, iodine, methanesulphonate or trifluoromethanesulphonate.

Most preferably X⁻ is a bromide anion. Thus, the most preferred anticholinergic according to the first embodiment of this invention is tiotropium bromide, including tiotropium bromide monohydrate, preferably in form of its crystalline monohydrate as disclosed in WO 02/30928. In case tiotropium bromid is used in anhydrous form, it is preferably present in form of the crystalline tiotropium bromide anhydrate disclosed in WO 03/0002.65.

According to a second preferred embodiment of this invention, the pharmaceutical composition comprises a compound of the formula 1b 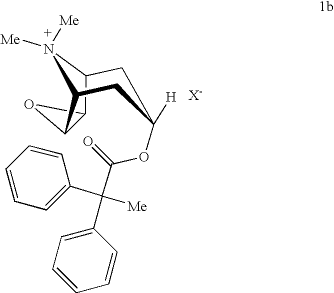
wherein
X⁻ represents chlorine, bromine, iodine, methanesulphonate or trifluoromethanesulphonate.
   Most preferably X⁻ is a bromide anion.
   The compounds of formula 1b are known in the art (WO 02/32899).

Hereinafter, compounds represented by the fomula 1 and 2 are referred to as Compound 1 and Compound 2, respectively. The compounds having the other formula numbers are referred to in the same manner.

Example of anticholinergics include following compounds (1)˜(L). 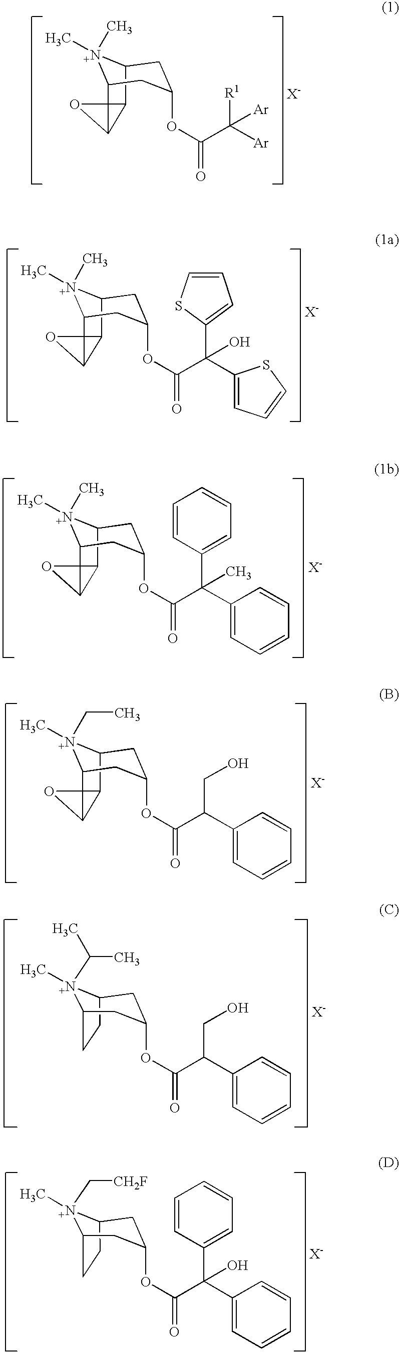 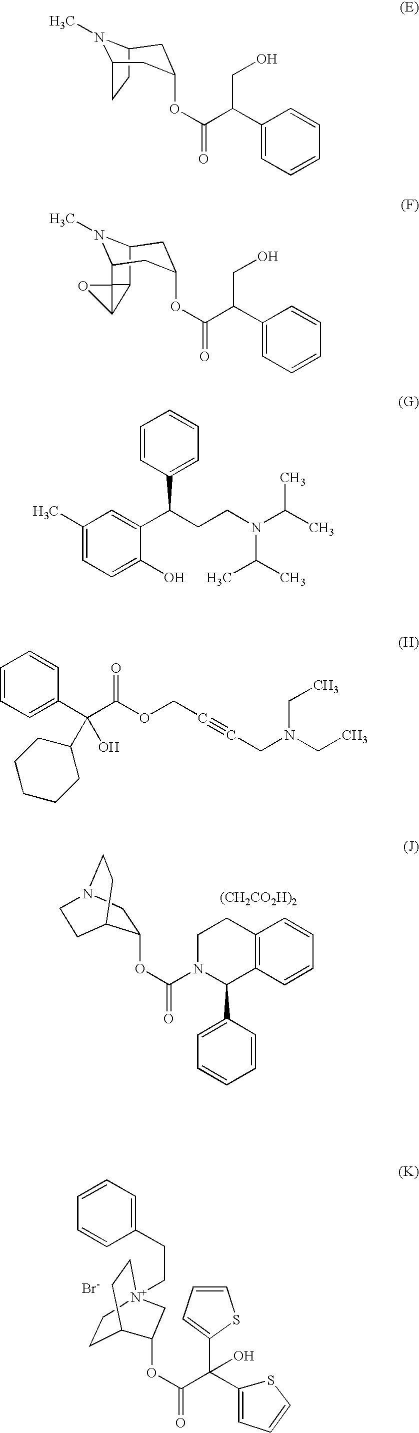 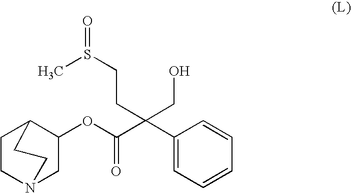
wherein
X⁻ represents chlorine, bromine, iodine, methanesulphonate or trifluoromethanesulphonate;
R¹ represents hydroxy or methyl;
Ar represents phenyl or thienyl, preferably 2-thienyl;

The pharmaceutical combinations of Compound 1 and Compound 2 according to the invention are preferably administered by inhalation. Suitable inhalable powders packed into suitable capsules (inhalettes) may be administered using suitable powder inhalers. Alternatively, the drug may be inhaled by the application of suitable inhalation aerosols. These also include inhalation aerosols which contain HFA134a (also known as TG134a), HFA227 (also known as TG227) or a mixture thereof as propellant gas. The drug may also be inhaled using suitable solutions of the pharmaceutical combination comprising of Compound 1 and Compound 2. The single dose to be administered to the patient may be achieved by actuating the inhaler once, twice, three or more times, preferably by actuating the inhaler once or twice, most preferably once.

The active substances may be combined in a single preparation or contained in two separate formulations. Pharmaceutical compositions in which Compound 1 and Compound 2 are combined in a single preparation are preferred according to the invention.

In another aspect the present invention relates to a pharmaceutical composition which contains, in addition to therapeutically effective quantities of Compound 1 and Compound 2, a pharmaceutically acceptable excipient. In another aspect the present invention relates to a pharmaceutical composition which does not contain any pharmaceutically acceptable excipient in addition to therapeutically effective quantites of Compound 1 and Compound 2.

The present invention also relates to the use of Compound 1 and Compound 2 for preparing a pharmaceutical composition containing therapeutically effective quantifies of Compound 1 and Compound 2 for treating inflammatory and/or obstructive diseases of the respiratory tract, particularly asthma or chronic obstructive pulmonary disease (COPD), and complications thereof such as pulmonary hypertension, as well as allergic and non-allergic rhinitis.

The present invention also relates to the use of a pharmaceutical formulation containing Compound 1 for preparing a pharmaceutical composition for treating inflammatory and/or obstructive diseases of the respiratory tract, particularly asthma or chronic obstructive pulmonary disease (COPD), as well as complications thereof such as pulmonary hypertension, as well as allergic and non-allergic rhinitis, characterised in that the pharmaceutical formulation contains Compound 2.

This present invention also relates to the prophylaxis of, treating of, or reducing the exacerbations associated with pulmonary diseases, in particular inflammatory or obstructive diseases of the respiratory tract, by administering to a patient in need thereof an effective amount of a pharmaceutical composition according to this invention either in a single combined form, separately, or separately and sequentially where the sequential administration is close in time, or remote in time.

In addition, the present invention also relates to the use of Compound 1 in the prophylaxis of, treating of, or reducing the exacerbations associated with pulmonary diseases, in particular inflammatory or obstructive diseases of the respiratory tract, by administering to a patent in need thereof an effective amount of Compound 1 in combination with Compound 2 either in a single combined form, separately, or separately and sequentially where the sequential administration is close in time, or remote in time.

Furthermore, the present invention also relates to the use of Compound 2 in the prophylaxis of, treating of, or reducing the exacerbations associated with pulmonary diseases, in particular inflammatory or obstructive diseases of the respiratory tract, by administering to a patient in need thereof an effective amount of Compound 1 in combination with Compound 2 either in a single combined form, separately, or separately and sequentially where the sequential administration is close in time, or remote in time.

Consequently, the present invention also relates to the use of Compound 1 in the manufacture of a medicament for the prophylaxis of, treating of, or reducing the exacerbations associated with pulmonary diseases, in particular inflammatory or obstructive diseases of the respiratory tract, by administering to a patient in need thereof an effective amount of Compound 1 in combination with Compound 2 either in a single combined form, separately, or separately and sequentially where the sequential administration is close in time, or remote in time.

Furthermore consequently, the present invention also relates to the use of Compound 2 in the manufacture of a medicament for the prophylaxis of, treating of, or reducing the exacerbations associated with pulmonary diseases, in particular inflammatory or obstructive diseases of the respiratory tract, by administering to a patient in need thereof an effective amount of Compound 1 in combination with Compound 2 either in a single combined form, separately, or separately and sequentially where the sequential administration is close in time, or remote in time.

The present invention also relates to the simultaneous or successive use of therapeutically effective doses of Compound 1 and Compound 2 for treating inflammatory and/or obstructive diseases of the respiratory tract, particularly asthma or chronic obstructive pulmonary disease (COPD), and complications thereof such as pulmonary hypertension, as well as allergic and non-allergic rhinitis, by simultaneous or successive administration.

The therapeutic utility of the combinations of compounds of the present invention is applicable to a patient or subject afflicted with a disease or condition as herein set forth and therefore in need of such treatment. The beneficial results are therapeutic whether administered to animals or humans. As used herein the terms “animal” and “animals” is used merely for the purpose of pointing out human beings as opposed to other members of the animal kingdom. The combinations of compounds of the present invention have therapeutic applicability in the treatment of mammals, and in particular of humans. All of the major subdivisions of the class of mammals (Mammalia) are included within the scope of the present invention with regard to being recipients of therapeutic treatment as described herein. Mammals have value as pets to humans and are therefore likely to be subjects of treatment. This applies especially to the canine and feline groups of mammals. Other mammals are valued as domesticated animals and their treatment in accordance with the present invention is likely in view of the adverse economic impact of not treating the diseases and conditions described herein. This applies especially to the equine, bovine, porcine, and ovine groups of mammals.

The pharmaceutical composition of the present invention is useful in the treatment and/or prophylaxis of obstructive airways diseases, especially of asthma, chronic obstructive pulmonary disease (COPD) and other obstructive airways diseases exacerbated by heightened bronchial reflexes, inflammation, bronchial hyper-reactivity and bronchospasm, in particular COPD.

In particular, the combination of Compound 1 and Compound 2 are useful in the treatment of respiratory diseases and conditions comprising: asthma, acute respiratory distress syndrome, chronic pulmonary inflammatory disease, bronchitis, chronic bronchitis, chronic obstructive pulmonary (airway) disease, including the pulmonary symptoms associated with cystic fibrosis and mucoviscidosis; and silicosis; or immune diseases and conditions comprising: allergic rhinitis and chronic sinusitis.

The types of diseases that may be treated using the combination of Compound 1 and Compound 2 include but are not limited to asthma; chronic or acute bronchoconstriction; chronic bronchitis; small airways obstruction; emphysema; chronic obstructive pulmonary disease (COPD); COPD that has chronic bronchitis, pulmonary emphysema or dyspnea associated therewith; COPD that is characterized by irreversible, progressive airways obstruction; adult respiratory distress syndrome (ARDS); exacerbation of airways hyper-reactivity consequent to drug therapy; pneumoconiosis; acute bronchitis; acute laryngotracheal bronchitis; arachidic bronchitis; catarrhal bronchitis; croupus bronchitis; dry bronchitis; infectious asthmatic bronchitis; productive bronchitis; staphylococcus or streptococcal bronchitis; vesicular bronchitis; cylindric bronchiectasis; sacculated bronchiectasis; fusiform bronchiectasis; capillary bronchiectasis; cystic bronchiectasis; dry bronchiectasis; follicular bronchiectasis; seasonal allergic rhinitis; perennial allergic rhinitis; purulent or nonpurulent sinusitis; acute or chronic sinusitis; ethmoid, frontal, maxillary, or sphenoid sinusitis; eosinophilia; pulmonary infiltration eosinophilia; Loffler's syndrome; chronic eosinophilic pneumonia; tropical pulmonary eosinophilia; bronchopneumonic aspergillosis; aspergilloma; granulomas containing eosinophils; allergic granulomatous angiitis or Churg-Strauss syndrome; sarcoidosis; alveolitis; chronic hypersensitivity pneumonitis; diffuse interstitial pulmonary fibrosis or interstitial lung fibrosis; and idiopathic pulmonary fibrosis.

One of the important respiratory diseases treatable with the combinations of therapeutic agents of the present invention is asthma, a chronic, increasingly common disorder encountered worldwide and characterized by intermittent reversible airway obstruction, airway hyper-responsiveness and inflammation. The cause of asthma has yet to be determined, but the most common pathological expression of asthma is inflammation of the airways, which may be significant even in the airways of patients with mild asthma. Based on bronchial biopsy and lavage studies it has been clearly shown that asthma involves infiltration by mast cells, eosinophils, and T-lymphocytes into a patient's airways. Bronchoalveolar lavage (BAL) in atopic asthmatics shows activation of interleukin (IL)-3, IL-4, IL-5 and granulocyte/macrophage-colony stimulating factor (GM-CSF) that suggests the presence of a T-helper 2 (Th-2)-like T-cell population.

The combinations of therapeutic agents of the present invention are useful in the treatment of atopic and non-atopic asthma. The term “atopy” refers to a genetic predisposition toward the development of type I (immediate) hypersensitivity reactions against common environmental antigens. The most common clinical manifestation is allergic rhinitis, while bronchial asthma, atopic dermatitis, and food allergy occur less frequently. Accordingly, the expression “atopic asthma” as used herein is intended to be synonymous with “allergic asthma”, i.e., bronchial asthma which is an allergic manifestation in a sensitized person. The term “non-atopic asthma” as used herein is intended to refer to all other asthmas, especially essential or “true” asthma, which is provoked by a variety of factors, including vigorous exercise, irritant particles, psychologic stresses, etc.

A single dose of the combination of Compound 1 and Compound 2 according to this invention is administered to the patient, preferably a human, in need of such treatment and/or prophylaxis once, twice, three times or more per day, preferably one or twice, most preferably once per day. Preferably Compound 1 and Compound 2 are co-administered, more preferably co-administered as a combined fixed dose, but these active substances may also be administered sequentially, whereby sequentially is preferably close in time, preferably the time gap between an administration of Compound 1 and Compound 2 or vice versa is 3 hours or less, more preferably 1 hour or less.

In the active substance combinations of Compound 1 and Compound 2 according to the invention, ingredients Compound 1 and Compound 2 may be present in the form of their enantiomers, mixtures of enantiomers or in the form of racemates.

The proportions in which Compound 1 and Compound 2 may be used in the active substance combinations according to the invention are variable. Compound 1 and Compound 2 may possibly be present in the form of their solvates or hydrates. Depending on the choice of Compound 1 and Compound 2, the weight ratios which may be used within the scope of the present invention vary on the basis of the different molecular weights of the various compounds and their different potencies.

As a rule, the pharmaceutical combinations according to the invention may contain Compound 1 and Compound 2 in ratios by weight ranging from 1:4000 to 8:1, preferably from 1:1000 to 1:1.2, more preferably from 1:1000 to 1:2,5, wherein the ratio is based on the weight of the corresponding cation 1 of Compound 1.

In the preferred combination of Compound 1a and Compound 2 according to this invention, the lower limit of the weight ratio of Compound 1a to Compound 2, calculated as the amount of the tiotropiumcation 1′ to Compound 2, is preferably 1:4000, more preferably 1:1000, most preferably 1:361; whereas the upper limit of the weight ratio of Compound 1a to Compound 2, calculated as the amount of the corresponding cation 1′ of Compound 1a to Compound 2, is preferably 1:2.5, more preferably 1:12.5, most preferably 1:24. The corresponding lower and upper limits with regard to tiotropium salts, especially tiotropium bromide, and/or hydrates, in particular tiotropium bromide monohydrate, can be calculated accordingly based on the corresponding molecular weights.

In the preferred combination of Compound 1b and Compound 2 according to this invention, the lower limit of the weight ratio of Compound 1b to Compound 2, calculated as the amount of the corresponding cation 1′ of Compound 1b to Compound 2, is preferably 1:4000, more preferably 1:1000, most preferably 1:361; whereas the upper limit of the weight ratio of Compound 1b to Compound 2, calculated as the amount of the corresponding cation 1′ of Compound 1b to Compound 2, is preferably 8:1, more preferably 1:1.2. The corresponding lower and upper limits with regard to salts of Compound 1b, especially the bromide, and/or hydrates can be calculated accordingly based on the corresponding molecular weights.

For example, without restricting the scope of the invention thereto, preferred combinations of Compound 1a and Compound 2 according to the invention may contain tiotropium 1′ and Compound 2 in the following weight ratios: 1:361, 1:350, 1:340, 1:330, 1:320, 1:310, 1:290, 1:280, 1:270, 1:260, 1:250, 1:240, 1:230, 1:220, 1:210, 1:200, 1:190, 1:180, 1:170, 1:160, 1:150, 1:140, 1:130, 1:120, 1:110, 1:100, 1:90, 1:80, 1:70, 1:60, 1:50, 1:40, 1:30, 1:24.

For example, without restricting the scope of the invention thereto, preferred combinations of Compound 1b and Compound 2 according to the invention may contain the cation 1′ of Compound 1b and Compound 2 in the following weight ratios: 1:361, 1:350, 1:340, 1:330, 1:320, 1:310, 1:290, 1:280, 1:270, 1:260, 1:250, 1:240, 1:230, 1:220, 1:210, 1:200, 1:190, 1:180, 1:170, 1:160, 1:150, 1:140, 1:130, 1:120, 1:110, 1:100, 1:90, 1:80, 1:70, 1:60, 1:50, 1:40, 1:30, 1:20, 1:10, 1:5, 1:3, 1:1.2.

The pharmaceutical compositions according to the invention containing the combinations of Compound 1 and Compound 2 are normally administered so that Compound 1 and Compound 2 are present together in doses of 25 to 10000 μg, preferably from 100 to 5800 μg, more preferably from 500 to 3400 μg per single dose.

Combinations of Compound 1a and Compound 2 according to the invention contain a quantity of tiotropium 1′ and Compound 2 such that the lower limit of the total dosage per single dose is preferably 27.5 μg, more preferably 105 μg, most preferably 508 μg; whereas the upper limit of the total dosage per single dose is preferably 10000 μg, more preferably 5040 μg, most preferably 3021 μg.

For example, combinations of Compound 1a and Compound 2 according to the invention contain a quantity of tiotropium 1′ and Compound 2 such that the total dosage per single dose is about 508 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1000 μg, 1050 μg, 1150 μg, 1100 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 2300 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg or similar. The suggested dosages per single dose specified above are not to be regarded as being limited to the numerical values actually stated, but are intended as dosages which are disclosed by way of example. Of course, dosages which fluctuate about the abovementioned numerical values within a range of about +/−25 μg are also included in the values given above by way of example. In these dosage ranges, the cation 1′ and Compound 2 may be present in the weight ratios given above.

Combinations of Compound 1b and Compound 2 according to the invention contain a quantity of the cation 1′ of Compound 1b and Compound 2 such that the lower limit of the total dosage per single dose is preferably 27.5 μg, more preferably 105 μg, most preferably 508 μg; whereas the upper limit of the total dosage per single dose is preferably 10800 μg, more preferably 5412 μg, most preferably 3412 μg.

For example, combinations of Compound 1b and Compound 2 according to the invention contain a quantity of the cation 1′ of Compound 1b and Compound 2 such that the total dosage per single dose is about 508 μg, 510 μg, 515 μg, 520 μg, 525 μg, 530 μg, 535 μg, 540 μg, 545 μg, 550 μg, 555 μg, 560 μg, 565 μg, 570 μg, 575 μg, 580 μg, 585 μg, 590 μg, 595 μg, 600 μg, 605 μg, 610 μg, 615 μg, 620 μg, 625 μg, 630 μg, 635 μg, 640 μg, 645 μg, 650 μg, 655 μg, 660 μg, 665 μg, 670 μg, 675 μg, 680 μg, 685 μg, 690 μg, 695 μg, 700 μg, 705 μg, 710 μg, 715 μg, 720 μg, 725 μg, 730 μg, 735 μg, 740 μg, 745 μg, 750 μg, 755 μg, 760 μg, 765 μg, 770 μg, 775 μg, 780 μg, 785 μg, 790 μg, 795 μg, 800 μg, 805 μg, 810 μg, 815 μg, 820 μg, 825 μg, 830 μg, 835 μg, 840 μg, 845 μg, 850 μg, 855 μg, 860 μg, 865 μg, 870 μg, 875 μg, 880 μg, 885 μg, 890 μg, 895 μg, 900 μg, 905 μg, 910 μg, 915 μg, 920 μg, 925 μg, 930 μg, 935 μg, 940 μg, 945 μg, 950 μg, 955 μg, 960 μg, 965 μg, 970 μg, 975 μg, 980 μg, 985 μg, 990 μg, 995 μg, 1000 μg, 1050 μg, 1150 μg, 1100 μg, 1200 μg, 1250 μg, 1300 μg, 1350 μg, 1400 μg, 1450 μg, 1500 μg, 1550 μg, 1600 μg, 1650 μg, 1700 μg, 1750 μg, 1800 μg, 1850 μg, 1900 μg, 1950 μg, 2000 μg, 2050 μg, 2100 μg, 2150 μg, 2200 μg, 2250 μg, 23000 μg, 2350 μg, 2400 μg, 2450 μg, 2500 μg, 2550 μg, 2600 μg, 2650 μg, 2700 μg, 2750 μg, 2800 μg, 2850 μg, 2900 μg, 2950 μg, 3000 μg, 3050 μg, 3100 μg, 3150 μg, 3200 μg, 3250 μg, 3300 μg, 3350 μg, 3400 μg, 3410 μg or similar. The suggested dosages per single dose specified above are not to be regarded as being limited to the numerical values actually stated, but are intended as dosages which are disclosed by way of example. Of course, dosages which fluctuate about the abovementioned numerical values within a range of about +/−25 μg are also included in the values given above by way of example. In these dosage ranges, the cation 1′ and Compound 2 may be present in the weight ratios given above.

In a combination of the active substances Compound 1a and Compound 2 according to this invention, the lower limit of the quantity of Compound 1a calculated as the amount of tiotropium 1′ administered per single dose is preferably 2.5 μg, more preferably 5 μg, most preferably 8.3 μg; whereas the upper limit of the quantity of Compound 1a calculated as the amount of tiotropium 1′ administered per single dose is preferably 40 μg, more preferably 20.8 μg.

With respect to certain diseases, especially such diseases where a thick mucus layer will impair penetration of the active substances to the target receptor, like e.g. cystic fibrosis, higher quantities of tiotropium, in particular up to about 80 μg may be beneficial. The corresponding lower and upper limits with regard to other tiotropium salts, especially tiotropium bromide, and/or hydrates, in particular tiotropium bromide hydrate, can be calculated accordingly based on the corresponding molecular weights.

In a combination of Compound 1a and Compound 2 according to this invention, the lower limit of the quantity of Compound 2 administered per single dose is preferably 25 μg, more preferably 100 μg, most preferably 500 μg; whereas the upper limit of the quantity of Compound 2 administered per single dose is preferably 10000 μg, more preferably 5000 μg, most preferably 3000 μg. The corresponding lower and upper limits with regard to other salts and/or hydrates of Compound 2 can be calculated accordingly based on the corresponding molecular weights.

For example, without restricting the scope of the invention thereto, according to the first embodiment of this invention the combinations of Compound 1a and Compound 2 may contain a quantity of tiotropium 1′ and Compound 2 such that, for each single dose, 8.3 μg of 1′ and 500 μg of Compound 2, 10 μg of 1′ and 500 μg of Compound 2, 12 μg of 1′ and 500 μg of Compound 2, 14 μg of 1′ and 500 μg of Compound 2, 16 μg of 1′ and 500 μg of Compound 2, 18 μg of 1′ and 500 μg of Compound 2, 20.8 μg of 1′ and 500 μg of Compound 2, 8.3 μg of 1′ and 750 μg of Compound 2, 10 μg of 1′ and 750 μg of Compound 2, 12 μg of 1′ and 750 μg of Compound 2, 14 μg of 1′ and 750 μg of Compound 2, 16 μg of 1′ and 750 μg of Compound 2, 18 μg of and 750 μg of Compound 2, 20.8 μg of 1′ and 750 μg of Compound 2,

8.3 μg of 1′ and 1000 μg of Compound 2, 10 μg of 1′ and 1000 μg of Compound 2, 12 μg of 1′ and 1000 μg of Compound 2, 14 μg of and 1000 μg of Compound 2, 16 μg of 1′ and 1000 μg of Compound 2, 18 μg of 1′ and 1000 μg of Compound 2, 20.8 μg of 1′ and 1000 μg of Compound 2,

8.3 μg of 1′ and 1250 μg of Compound 2, 10 μg of 1′ and 1250 μg of Compound 2, 12 μg of 1′ and 1250 μg of Compound 2, 14 μg of 1′ and 1250 μg of Compound 2, 16 μg of 1′ and 1250 μg of Compound 2, 18 μg of 1′ and 1250 μg of Compound 2, 20.8 μg of 1′ and 1250 μg of Compound 2,

8.3 μg of 1′ and 1500 μg of Compound 2, 10 μg of 1′ and 1500 μg of Compound 2, 12 μg of 1′ and 1500 μg of Compound 2, 14 μg of 1′ and 1500 μg of Compound 2, 16 μg of 1′ and 1500 μg of Compound 2, 18 μg of 1′ and 1500 μg of Compound 2, 20.8 μg of 1′ and 1500 μg of Compound 2,

8.3 μg of 1′ and 1750 μg of Compound 2, 10 μg of 1′ and 1750 μg of Compound 2, 12 μg of 1′ and 1750 μg of Compound 2, 14 μg of 1′ and 1750 μg of Compound 2, 16 μg of 1′ and 1750 μg of Compound 2, 18 μg of 1′ and 1750 μg of Compound 2, 20.8 μg of 1′ and 1750 μg of Compound 2,

8.3 μg of 1′ and 2000 μg of Compound 2, 10 μg of 1′ and 2000 μg of Compound 2, 12 μg of 1′ and 2000 μg of Compound 2, 14 μg of 1′ and 2000 μg of Compound 2, 16 μg of 1′ and 2000 μg of Compound 2, 18 μg of 1′ and 2000 μg of Compound 2, 20.8 μg of 1′ and 2000 μg of Compound 2,

8.3 μg of 1′ and 2250 μg of Compound 2, 10 μg of 1′ and 2250 μg of Compound 2, 12 μg of 1′ and 2250 μg of Compound 2, 14 μg of 1′ and 2250 μg of Compound 2, 16 μg of 1′ and 2250 μg of Compound 2, 18 μg of 1′ and 2250 μg of Compound 2, 20.8 μg of 1′ and 2250 μg of Compound 2,

8.3 μg of 1′ and 2500 μg of Compound 2, 10 μg of and 2500 μg of Compound 2, 12 μg of 1′ and 2500 μg of Compound 2, 14 μg of 1′ and 2500 μg of Compound 2, 16 μg of 1′ and 2500 μg of Compound 2, 18 μg of 1′ and 2500 μg of Compound 2, 20.8 μg of 1′ and 2500 μg of Compound 2,

8.3 μg of 1′ and 2750 μg of Compound 2, 10 μg of 1′ and 2750 μg of Compound 2, 12 μg of 1′ and 2750 μg of Compound 2, 14 μg of 1′ and 2750 μg of Compound 2, 16 μg of 1′ and 2750 μg of Compound 2, 18 μg of and 2750 μg of Compound 2, 20.8 μg of 1′ and 2750 μg of Compound 2,

8.3 μg of 1′ and 3000 μg of Compound 2, 10 μg of 1′ and 3000 μg of Compound 2, 12 μg of 1′ and 3000 μg of Compound 2, 14 μg of 1′ and 3000 μg of Compound 2, 16 μg of 1′ and 3000 μg of Compound 2, 18 μg of 1′ and 3000 μg of Compound 2, 20.8 μg of 1′ and 3000 μg of Compound 2 are administered.

The corresponding quantifies with regard to tiotropium salts Compound 1a, especially tiotropium bromide, and/or hydrates, in particular tiotropium bromide monohydrate, can be calculated accordingly based on the corresponding molecular weights.

In a combination of Compound 1b and Compound 2 according to this invention, the lower limit of the quantity of Compound 1b calculated as the amount of the corresponding cation 1′ administered per single dose is preferably 2.5 μg, more preferably 5 μg, most preferably 8.3 μg; whereas the upper limit of the quantity of Compound 1b calculated as the amount of the corresponding cation 1′ of Compound 1 administered per single dose is preferably 800 μg, more preferably 412.8 μg. The corresponding lower and upper limits with regard to salts of Compound 1b, especially the bromide, and/or hydrates can be calculated accordingly based on the corresponding molecular weights.

In a combination of Compound 1b and Compound 2 according to this invention, the lower limit of the quantity of Compound 2 administered per single dose is preferably 25 μg, more preferably 100 μg, most preferably 500 μg; whereas the upper limit of the quantity of Compound 2 administered per single dose is preferably 10000 μg, more preferably 5000 μg, most preferably 3000 μg. The corresponding lower and upper limits with regard to other salts and/or hydrates of Compound 2 can be calculated accordingly based on the corresponding molecular weights.

For example and without restricting the scope of the invention thereto, according to the second embodiment of this invention the combinations of Compound 1b and Compound 2 may contain an amount of the corresponding cation 1′ of Compound 1 b and Compound 2 such that 8.3 μg of 1′ and 25 μg of Compound 2, 8.3 μg of 1′ and 50 μg of Compound 2, 8.3 μg of 1′ and 100 μg of Compound 2, 8.3 μg of 1′ and 200 μg of Compound 2, 8.3 μg of 1′ and 300 μg of Compound 2, 8.3 μg of 1′ and 400 μg of Compound 2, 8.3 μg of 1′ and 500 μg of Compound 2, 8.3 μg of 1′ and 600 μg of Compound 2, 8.3 μg of 1′ and 700 μg of Compound 2, 8.3 μg of 1′ and 800 μg of Compound 2, 8.3 μg of 1′ and 900 μg of Compound 2, 8.3 μg of 1′ and 1000 μg of Compound 2, 8.3 μg of 1′ and 1100 μg of Compound 2, 8.3 μg of 1′ and 1200 μg of Compound 2, 8.3 μg of 1′ and 1300 μg of Compound 2, 8.3 μg of 1′ and 1400 μg of Compound 2, 8.3 μg of 1′ and 1500 μg of Compound 2, 8.3 μg of 1′ and 1600 μg of Compound 2, 8.3 μg of 1′ and 1700 μg f Compound 2, 8.3 μg of 1′ and 1800 μg of Compound 2, 8.3 μg of 1′ and 1900 μg of Compound 2, 8.3 μg of 1′ and 2000 μg of Compound 2, 8.3 μg of 1′ and 2100 μg of Compound 2, 8.3 μg of 1′ and 2200 μg of Compound 2, 8.3 μg of 1′ and 2300 μg of Compound 2, 8.3 μg of 1′ and 2400 μg of Compound 2, 8.3 μg of 1′ and 2500 μg of Compound 2, 8.38 μg of 1 and 2600 μg of Compound 2, 8.3 μg of 1′ and 2700 μg of Compound 2, 8.3 μg of 1′ and 2800 μg of Compound 2, 8.3 μg of 1′ and 2900 μg of Compound 2, 8.3 μg of 1′ and 3000 μg of Compound 2,

16.5 μg of 1′ and 25 μg of Compound 2, 16.5 μg of 1′ and 50 μg of Compound 2, 16.5 μg of 1′ and 100 μg of Compound 2, 16.5 μg of and 200 μg of Compound 2, 16.5 μg of 1′ and 300 μg of Compound 2, 16.5 μg of 1′ and 400 μg of Compound 2, 16.5 μg of 1′ and 500 μg of Compound 2, 16.5 μg of 1′ and 600 μg of Compound 2, 16.5 μg of 1′ and 700 μg of Compound 2, 16.5 μg of 1′ and 800 μg of Compound 2, 16.5 μg of 1′ and 900 μg of Compound 2, 16.5 μg of 1′ and 1000 μg of Compound 2, 16.5 μg of 1′ and 1100 μg of Compound 2, 16.5 μg of 1′ and 1200 μg of Compound 2, 16.5 μg of 1′ and 1300 μg of Compound 2, 16.5 μg of 1′ and 1400 μg of Compound 2, 16.5 μg of 1′ and 1500 μg of Compound 2, 16.5 μg of 1′ and 1600 μg of Compound 2, 16.5 μg of 1′ and 1700 μg of Compound 2, 16.5 μg of 1′ and 1800 μg of Compound 2, 16.5 μg of 1′ and 1900 μg of Compound 2, 16.5 μg of 1′ and 2000 μg of Compound 2, 16.5 μg of 1′ and 2100 μg of Compound 2, 16.5 μg of 1′ and 2200 μg of Compound 2, 16.5 μg of 1′ and 2300 μg of Compound 2, 16.5 μg of 1′ and 2400 μg of Compound 2, 16.5 μg of 1′ and 2500 μg of Compound 2, 16.5 μg of 1′ and 2600 μg of Compound 2, 16.5 μg of 1′ and 2700 μg of Compound 2, 16.5 μg of 1′ and 2800 μg of Compound 2, 16.5 μg of 1′ and 2900 μg of Compound 2, 16.5 μg of 1′ and 3000 μg of Compound 2,

33 μg of 1′ and 25 μg of Compound 2, 33 μg of 1′ and 50 μg of Compound 2, 33 μg of 1′ and 100 μg of Compound 2, 33 μg of 1′ and 200 μg of Compound 2, 33 μg of 1′ and 300 μg of Compound 2, 33 μg of 1′ and 400 μg of Compound 2, 33 μg of 1′ and 500 μg of Compound 2, 33 μg of 1′ and 600 μg of Compound 2, 33 μg of 1′ and 700 μg of Compound 2, 33 μg of 1′ and 800 μg of Compound 2, 33 μg of 1′ and 900 μg of Compound 2, 33 μg of 1′ and 1000 μg of Compound 2, 33 μg of 1′ and 1100 μg of Compound 2, 33 μg of 1′ and 1200 μg of Compound 2, 33 μg of 1′ and 1300 μg of Compound 2, 33 μg of 1′ and 1400 μg of Compound 2, 33 μg of 1 and 1500 μg of Compound 2, 33 μg of 1′ and 1600 μg of Compound 2, 33 μg of 1′ and 1700 μg of Compound 2, 33 μg of 1′ and 1800 μg of Compound 2, 33 μg of 1′ and 1900 μg of Compound 2, 33 μg of 1′ and 2000 μg of Compound 2, 33 μg of 1′ and 2100 μg of Compound 2, 33 μg of 1′ and 2200 μg of Compound 2, 33 μg of 1′ and 2300 μg of Compound 2, 33 μg of 1′ and 2400 μg of Compound 2, 33 μg of 1′ and 2500 μg of Compound 2, 33 μg of 1′ and 2600 μg of Compound 2, 33 μg of 1′ and 2700 μg of Compound 2, 33 μg of 1′ and 2800 μg of Compound 2, 33 μg of 1′ and 2900 μg of Compound 2, 33 μg of 1′ and 3000 μg of Compound 2,

49.5 μg of 1′ and 25 μg of Compound 2, 49.5 μg of 1′ and 50 μg of Compound 2, 49.5 μg of 1′ and 100 μg of Compound 2, 49.5 μg of 1′ and 200 μg of Compound 2, 49.5 μg of 1′ and 300 μg of Compound 2, 49.5 μg of 1′ and 400 μg of Compound 2, 49.5 μg of 1′ and 500 μg of Compound 2, 49.5 μg of 1′ and 600 μg of Compound 2, 49.5 μg of 1′ and 700 μg of Compound 2, 49.5 μg of 1′ and 800 μg of Compound 2, 49.5 μg of 1′ and 900 μg of Compound 2, 49.5 μg of 1′ and 1000 μg of Compound 2, 49.5 μg of 1′ and 1100 μg of Compound 2, 49.5 μg of 1′ and 1200 μg of Compound 2, 49.5 μg of 1′ and 1300 μg of Compound 2, 49.5 μg of 1′ and 1400 μg of Compound 2, 49.5 μg of 1′ and 1500 μg of Compound 2, 49.5 μg of 1′ and 1600 μg of Compound 2, 49.5 μg of 1′ and 1700 μg of Compound 2, 49.5 μg of 1 and 1800 μg of Compound 2, 49.5 μg of 1′ and 1900 μg of Compound 2, 49.5 μg of 1′ and 2000 μg of Compound 2, 49.5 μg of 1′ and 2100 μg of Compound 2, 49.5 μg of 1′ and 2200 μg of Compound 2, 49.5 μg of 1′ and 2300 μg of Compound 2, 49.5 μg of 1′ and 2400 μg of Compound 2, 49.5 μg of 1′ and 2500 μg of Compound 2, 49.5 μg of 1′ and 2600 μg of Compound 2, 49.5 μg of 1′ and 2700 μg of Compound 2, 49.5 μg of 1′ and 2800 μg of Compound 2, 49.5 μg of 1′ and 2900 μg of Compound 2, 49.5 μg of 1′ and 3000 μg of Compound 2,

82.6 μg of 1′ and 25 μg of Compound 2, 82.6 μg of 1′ and 50 μg of Compound 2, 82.6 μg of 1′ and 100 μg of Compound 2, 82.6 μg of 1′ and 200 μg of Compound 2, 82.6 μg of 1′ and 300 μg of Compound 2, 82.6 μg of 1′ and 400 μg of Compound 2, 82.6 μg of 1′ and 500 μg of Compound 2, 82.6 μg of 1 and 600 μg of Compound 2, 82.6 μg of 1′ and 700 μg of Compound 2, 82.6 μg of 1′ and 800 μg of Compound 2, 82.6 μg of 1′ and 900 μg of Compound 2, 82.6 μg of 1′ and 1000 μg of Compound 2, 82.6 μg of 1′ and 1100 μg of Compound 2, 82.6 μg of 1′ and 1200 μg of Compound 2, 82.6 μg of v and 1300 μg of Compound 2, 82.6 μg of 1′ and 1400 μg of Compound 2, 82.6 μg of 1′ and 1500 μg of Compound 2, 82.6 μg of 1′ and 1600 μg of Compound 2, 82.6 μg of 1′ and 1700 μg of Compound 2, 82.6 μg of 1′ and 1800 μg of Compound 2, 82.6 μg of 1′ and 1900 μg of Compound 2, 82.6 μg of 1′ and 2000 μg of Compound 2, 82.6 μg of 1′ and 2100 μg of Compound 2, 82.6 μg of 1′ and 2200 μg of Compound 2, 82.6 μg of 1′ and 2300 μg of Compound 2, 82.6 μg of 1′ and 2400 μg of Compound 2, 82.6 μg of 1′ and 2500 μg of Compound 2, 82.6 μg of 1′ and 2600 μg of Compound 2, 82.6 μg of 1′ and 2700 μg of Compound 2, 82.6 μg of 1′ and 2800 μg of Compound 2, 82.6 μg of 1′ and 2900 μg of Compound 2, 82.6 μg of 1′ and 3000 μg of Compound 2,

16.5 μg of 1′ and 25 μg of Compound 2, 165.1 μg of 1′ and 50 μg of Compound 2, 165.1 μg of 1′ and 100 μg of Compound 2, 165.1 μg of 1′ and 200 μg of Compound 2, 165.1 μg of 1′ and 300 μg of Compound 2, 165.1 μg of 1′ and 400 μg of Compound 2, 165.1 μg of 1′ and 500 μg of Compound 2, 165.1 μg of 1′ and 600 μg of Compound 2, 165.1 μg of 1′ and 700 μg of Compound 2, 165.1 μg of 1′ and 800 μg of Compound 2, 165.1 μg of 1′ and 900 μg of Compound 2, 165.1 μg of 1′ and 1000 μg of Compound 2, 165.1 μg of 1′ and 1100 μg of Compound 2, 165.1 μg of 1′ and 1200 μg of Compound 2, 165.1 μg of 1′ and 1300 μg of Compound 2, 165.1 μg of 1′ and 1400 μg of Compound 2, 165.1 μg of 1′ and 1500 μg of Compound 2, 165.1 μg of 1′ and 1600 μg of Compound 2, 165.1 μg of 1′ and 1700 μg of Compound 2, 165.1 μg of 1′ and 1800 μg of Compound 2, 165.1 μg of 1′ and 1900 μg of Compound 2, 165.1 μg of 1′ and 2000 μg of Compound 2, 165.1 μg of 1′ and 2100 μg of Compound 2, 165.1 μg of 1′ and 2200 μg of Compound 2, 165.1 μg of 1′ and 2300 μg of Compound 2, 165.1 μg of 1′ and 2400 μg of Compound 2, 165.1 μg of 1 and 2500 μg of Compound 2, 165.1 μg of 1′ and 2600 μg of Compound 2, 165.1 μg of 1′ and 2700 μg of Compound 2, 165.1 μg of 1′ and 2800 μg of Compound 2, 165.1 μg of 1′ and 2900 μg of Compound 2, 165.1 μg of 1′ and 3000 μg of Compound 2,

206,4 μg of 1′ and 25 μg of Compound 2, 206.4 μg of 1′ and 50 μg of Compound 2, 206.4 μg of 1′ and 100 μg of Compound 2, 206.4 μg of 1′ and 200 μg of Compound 2, 206.4 μg of 1′ and 300 μg of Compound 2, 206.4 μg of 1′ and 400 μg of Compound 2, 206.4 μg of 1′ and 500 μg of Compound 2, 206.4 μg of 1′ and 600 μg of Compound 2, 206.4 μg of 1′ and 700 μg of Compound 2, 206.4 μg of 1′ and 800 μg of Compound 2, 206.4 μg of 1′ and 900 μg of Compound 2, 206.4 μg of 1′ and 1000 μg of Compound 2, 206.4 μg of 1′ and 1100 μg of Compound 2, 206.4 μg of 1′ and 1200 μg of Compound 2, 206.4 μg of 1′ and 1300 μg of Compound 2, 206.4 μg of 1′ and 1400 μg of Compound 2, 206.4 μg of 1′ and 1500 μg of Compound 2, 206.4 μg of 1′ and 1600 μg of Compound 2, 206.4 μg of 1′ and 1700 μg of Compound 2, 206.4 μg of 1′ and 1800 μg of Compound 2, 206.4 μg of 1′ and 1900μg of Compound 2, 206.4 μg of 1′ and 2000 μg of Compound 2, 206.4 μg of 1′ and 2100 μg of Compound 2, 206.4 μg of 1′ and 2200 μg of Compound 2, 206.4 μg of 1′ and 2300 μg of Compound 2, 206.4 μg of 1′ and 2400 μg of Compound 2, 206.4 μg of 1′ and 2500 μg of Compound 2, 206.4 μg of 1′ and 2600 μg of Compound 2, 206.4 μg of 1′ and 2700 μg of Compound 2, 206.4 μg of 1′ and 2800 μg of Compound 2, 206.4 μg of 1′ and 2900 μg of Compound 2, 206.4 μg of 1′ and 3000 μg of Compound 2,

412.8 μg of 1′ and 25 μg of Compound 2, 412.8 μg of 1′ and 50 μg of Compound 2, 412.8 μg of 1′ and 100 μg of Compound 2, 412.8 μg of 1′ and 200 μg of Compound 2, 412.8 μg of 1′ and 300 μg of Compound 2, 412.8 μg of 1′ and 400 μg of Compound 2, 412.8 μg of 1′ and 500 μg of Compound 2 or 412.8 μg of 1′ and 600 μg of Compound 2, 412.8 μg of 1′ and 700 μg of Compound 2, 412.8 μg of 1′ and 800 μg of Compound 2, 412.8 μg of 1′ and 900 μg of Compound 2, 412.8 μg of 1′ and 1000 μg of Compound 2, 412.8 μg of 1′ and 1100 μg of Compound 2, 412.8 μg of 1′ and 1200 μg of Compound 2, 412.8 μg of 1′ and 1300 μg of Compound 2, 412.8 μg of 1′ and 1400 μg of Compound 2, 412.8 μg of 1′ and 1500 μg of Compound 2, 412.8 μg of 1′ and 1600 μg of Compound 2, 412.8 μg of 1′ and 1700 μg of Compound 2, 412.8 μg of 1′ and 1800 μg of Compound 2, 412.8 μg of 1′ and 1900 μg of Compound 2, 412.8 μg of 1′ and 2000 μg of Compound 2, 412.8 μg of 1′ and 2100 μg of Compound 2, 412.8 μg of 1′ and 2200 μg of Compound 2, 412.8 μg of 1′ and 2300 μg of Compound 2, 412.8μg of 1′ and 2400μg of Compound 2, 412.8 μg of 1′ and 2500 μg of Compound 2, 412.8 μg of 1′ and 2600 μg of Compound 2, 412.8 μg of 1′ and 2700 μg of Compound 2, 412.8 μg of 1′ and 2800 μg of Compound 2, 412.8 μg of 1′ and 2900 μg of Compound 2, 412.8 μg of 1′ and 3000 μg of Compound 2, are administered per single dose. The corresponding quantities with regard to salts of Compound 1b, especially the bromide, and/or hydrates can be calculated accordingly based on the corresponding molecular weights.

The active substance combinations of Compound 1 and Compound 2 according to the invention are preferably administered by inhalation. For this purpose, Compound 1 and Compound 2 have to be made available in forms suitable for inhalation. Inhalable preparations include inhalable powders, propellant-containing metering aerosols or propellant-free inhalable solutions. Inhalable powders according to the invention containing the combination of Compound 1 and Compound 2 may consist of the active substances on their own or of a mixture of the active substances with physiologically acceptable excipients. Within the scope of the present invention, the term propellant-free inhalable solutions also include concentrates or sterile inhalable solutions ready for use. The preparations according to the invention may contain the combination of Compound 1 and Compound 2 either together in one formulation or in two separate formulations. These formulations which may be used within the scope of the present invention are described in more detail in the next part of the specification.

A) Inhalable Powder Containing the Combinations of Compound 1 and Compound 2 According to the Invention:

The inhalable powders according to the invention may contain Compound 1 and Compound 2 either on their own or in admixture with suitable physiologically acceptable excipients.

If Compound 1 and Compound 2 are present in admixture with physiologically acceptable excipients, the following physiologically acceptable excipients may be used to prepare these inhalable powders according to the invention: monosaccharides (e.g. glucose or arabinose), disaccharides (e.g. lactose, saccharose, maltose, trehalose), oligo- and polysaccharides (e.g. dextran), cyclodextrines (e.g. α-cyclodextrine, β-cyclodextrine, x-cyclodextrine, methyl-β-cyclodextrine, hydroxypropyl-β-cyclodextrine), polyalcohols (e.g. sorbitol, mannitol, xylitol), salts (e.g. sodium chloride, calcium carbonate) or mixtures of these excipients with one another. Preferably, mono- or disaccharides are used, while the use of lactose or glucose is preferred, particularly, but not exclusively, in the form of their hydrates. For the purposes of the invention, lactose is the particularly preferred excipient, while lactose monohydrate is most particularly preferred.

Within the scope of the inhalable powders according to the invention the excipients have a maximum average particle size of up to 250 μm, preferably between 10 and 150 μm, most preferably between 15 and 80 μm. It may sometimes seem appropriate to add finer excipient fractions with ah average particle size of 1 to 9 μm to the excipient mentioned above. These finer excipients are also selected from the group of possible excipients listed hereinbefore. Finally, in order to prepare the inhalable powders according to the invention, micronised Compound 1 and Compound 2, preferably with an average particle size of 0.5 to 10 μm, more preferably from 1 to 5 μm, is added to the excipient mixture. Processes for producing the inhalable powders according to the invention by grinding and micronising and finally mixing the ingredients together are known from the prior art. The inhalable powders according to the invention may be prepared and administered either in the form of a single powder mixture which contains both Compound 1 and Compound 2 or in the form of separate inhalable powders which contain only Compound 1 or Compound 2.

The inhalable powders according to the invention may be administered using inhalers known from the prior art. Inhalable powders according to the invention which contain a physiologically acceptable excipient in addition to Compound 1 and Compound 2 may be administered, for example, by means, of inhalers which deliver a single dose from a supply using a measuring chamber as described in U.S. Pat. No. 4,570,630A, or by other means as described in DE 36 25 685 A. Preferably, the inhalable powders according to the invention which contain physiologically acceptable excipient in addition to Compound 1 and Compound 2 are packed into capsules (to produce so-called inhalettes) which are used in inhalers as described, for example, in WO 94/28958.

A particularly preferred inhaler for using the pharmaceutical combination according to the invention in inhalettes is shown in FIG. 1.

This inhaler (Handyhaler) for inhaling powdered pharmaceutical compositions from capsules is characterised by a housing 1 containing two windows 2, a deck 3 in which there are air inlet ports and which is provided with a screen 5 secured via a screen housing 4, an inhalation chamber 6 connected to the deck 3 on which there is a push button 8 provided with two sharpened pins 7 and movable counter to a spring 8, and a mouthpiece 12 which is connected to the housing 1, the deck 3 and a cover 11 via a spindle 10 to enable it to be flipped open or shut, as well as air holes 13 for adjusting the flow resistance.

If the inhalable powders according to the invention are packed into capsules (inhalers) for the preferred use described above, the quantities packed into each capsule should be 1 to 30 mg, preferably 3 to 20 mg, more particularly 5 to 10 mg of inhalable powder per capsule. These capsules contain, according to the invention, either together or separately, the doses of 1′ and Compound 2 mentioned hereinbefore for each single dose.

B) Propellant Gas-Driven Inhalation Aerosols Containing the Combinations of Compound 1 and Compound 2:

Inhalation aerosols containing propellant gas according to the invention may contain Compound 1 and Compound 2 dissolved in the propellant gas or in dispersed form.

Compound 1 and Compound 2 may be present in separate formulations or in a single preparation, in which Compound 1 and Compound 2 are either both dissolved, both dispersed or only one component is dissolved and the other is dispersed. The propellant gases which may be used to prepare the inhalation aerosols according to the invention are known from the prior art. Suitable propellant gases are selected from among hydrocarbons such as n-propane, n-butane or isobutane and halohydrocarbons such as preferably fluorinated derivatives of methane, ethane, propane, butane, cyclopropane or cyclobutane. The propellant gases mentioned above may be used on their own or in mixtures thereof. Particularly preferred propellant gases are halogenated alkane derivatives selected from TG11, TG12, TG134a (1,1,1,2-tetrafluoroethane) and TG227 (1,1,1,2,3,3,3-heptafluoropropane) and mixtures thereof, of which the propellant gases TG134a, TG227 and mixtures thereof are preferred.

The propellant-driven inhalation aerosols according to the invention may also contain other ingredients such as co-solvents, stabilisers, surfactants, antioxidants, lubricants and pH adjusters. All these ingredients are known in the art.

The inhalation aerosols containing propellant gas according to the invention may contain up to 5 wt.-% of Compound 1 and/or Compound 2. Aerosols according to the invention contain, for example, 0.002 to 5 wt.-%, 0.01 to 3 wt.-%, 0.015 to 2 wt.-%, 0.1 to 2 wt.-%, 0.5 to 2 wt.-% or 0.5 to 1 wt.-% of Compound 1 and/or Compound 2.

If Compound 1 and/or Compound 2 are present in dispersed form, the particles of active substance preferably have an average particle size of up to 10 μm, preferably from 0.1 to 5 μm, more preferably from 1 to 5 μm.

The propellant-driven inhalation aerosols according to the invention mentioned above may be administered using inhalers known in the art (MDIs=metered dose inhalers). Accordingly, in another aspect, the present invention relates to pharmaceutical compositions in the form of propellant-driven aerosols as hereinbefore described combined with one or more inhalers suitable for administering these aerosols. In addition, the present invention relates to inhalers which are characterised in that they contain the propellant gas-containing aerosols described above according to the invention.

The present invention also relates to cartridges which are fitted with a suitable valve and can be used in a suitable inhaler and which contain one of the above-mentioned propellant gas-containing inhalation aerosols according to the invention. Suitable cartridges and methods of filling these cartridges with the inhalable aerosols containing propellant gas according to the invention are known from the prior art.

C) Propellant-Free Inhalable Solutions or Suspensions Containing the Combinations of Compound 1 and Compound 2 According to the Invention:

Propellant-free inhalable solutions and suspensions according to the invention contain, for example, aqueous or alcoholic, preferably ethanolic solvents, optionally ethanolic solvents mixed with aqueous solvents. If aqueous/ethanolic solvent mixtures are used the relative proportion of ethanol compared with water is not limited but the maximum is up to 70 percent by volume, more particularly up to 60 percent by volume and most preferably up to 30 percent by volume. The remainder of the volume is made up of water. The solutions or suspensions containing Compound 1 and Compound 2, separately or together, are adjusted to a pH of 2 to 7, preferably 2 to 5, using suitable acids. The pH may be adjusted using acids selected from inorganic or organic acids. Examples of suitable inorganic acids include hydrochloric acid, hydrobromic acid, nitric acid, sulphuric acid and/or phosphoric acid. Examples of particularly suitable organic acids include ascorbic acid, citric acid, malic acid, tartaric acid, maleic acid, succinic acid, fumaric acid, acetic acid, formic acid and/or propionic acid etc. Preferred inorganic adds are hydrochloric and sulphuric acids. It is also possible to use the acids which have already formed an acid addition salt with one of the active substances. Of the organic adds, ascorbic acid, fumaric acid and citric acid are preferred. If desired, mixtures of the above acids may be used, particularly in the case of acids which have other properties in addition to their acidifying qualities, e.g. as flavourings, antioxidants or complexing agents, such as citric acid or ascorbic acid, for example. According to the invention, it is particularly preferred to use hydrochloric acid to adjust the pH.

According to the invention, the addition of editic acid (EDTA) or one of the known salts thereof, sodium edetate, as stabiliser or complexing agent is unnecessary in the present formulation. Other embodiments may contain this compound or these compounds. In a preferred embodiment the content based on sodium edetate is less than 100 mg/100 ml, preferably less than 50 mg/100 ml, more preferably less than 20 mg/100 ml. Generally, inhalable solutions in which the content of sodium edetate is from 0 to 10 mg/100 ml are preferred.

Co-solvents and/or other excipients may be added to the propellant-free inhalable solutions according to the invention. Preferred co-solvents are those which contain hydroxyl groups or other polar groups, e.g. alcohols—particularly isopropyl alcohol, glycols—particularly propyleneglycol, polyethyleneglycol, polypropyleneglycol, glycolether, glycerol, polyoxyethylene alcohols and polyoxyethylene fatty acid esters. The terms excipients and additives in this context denote any pharmacologically acceptable substance which is not an active substance but which can be formulated with the active substance or substances in the pharmacologically suitable solvent in order to improve the qualitative properties of the active substance formulation. These substances may have a pharmacological effect or not, but at least no undesirable pharmacological effect. The excipients and additives include, for example, surfactants such as soya lecithin, oleic acid, sorbitan esters, such as polysorbates, polyvinylpyrrolidone, other stabilisers, complexing agents, antioxidants and/or preservatives which guarantee or prolong the shelf life of the finished pharmaceutical formulation, flavourings, vitamins and/or other additives known in the art. The additives also include pharmacologically acceptable salts such as sodium chloride as isotonic agents.

The preferred excipients include antioxidants such as ascorbic acid, for example, provided that it has not already been used to adjust the pH, vitamin A, vitamin E, tocopherols and similar vitamins and provitamins occurring in the human body.

Preservatives may be used to protect the formulation from contamination with pathogens. Suitable preservatives are those which are known in the art, particularly cetyl pyridinium chloride, benzalkonium chloride or benzoic acid or benzoates such as sodium benzoate in the concentration known from the prior art. The preservatives mentioned above are preferably present in concentrations of up to 50 mg/100 ml, more preferably between 5 and 20 mg/100 ml.

Preferred formulations contain, in addition to the solvent water and the combination of Compound 1 and Compound 2, only benzalkonium chloride and sodium edetate. In another preferred embodiment, no sodium edetate is present.

The propellant-free inhalable solutions according to the invention are administered in particular using inhalers of the kind which are capable of nebulising a small amount of a liquid formulation in the therapeutic dose within a few seconds to produce an aerosol suitable for therapeutic inhalation. Within the scope of the present invention, preferred inhalers are those in which a quantity of less than 100 μL, preferably less than 50 μL, more preferably between 10 and 30 μL of active substance solution can be nebulised in preferably one spray action to form an aerosol with an average particle size of less than 20 μm, preferably less than 10 μm, in such a way that the inhalable part of the aerosol corresponds to the therapeutically effective quantity.

An apparatus of this kind for propellant-free delivery of a metered quantity of a liquid pharmaceutical composition for inhalation is described for example in International Patent Application WO 91/14468 and also in WO 97/12687 (cf. in particular FIGS. 6a and 6b). The nebulisers (devices) described therein are known by the name Respimat®.

This nebuliser (Respimat®) can advantageously be used to produce the inhalable aerosols according to the invention containing the combination of Compound 1 and Compound 2. Because of its cylindrical shape and handy size of less than 9 to 15 cm long and 2 to 4 cm wide, this device can be carried at all times by the patient. The nebuliser sprays a defined volume of pharmaceutical formulation using high pressures through small nozzles so as to produce inhalable aerosols.

The preferred atomiser essentially consists of an upper housing part, a pump housing, a nozzle, a locking mechanism, a spring housing, a spring and a storage container, characterised by
a pump housing which is secured in the upper housing part and which comprises at one end a nozzle body with the nozzle or nozzle arrangement,
a hollow plunger with valve body,
a power takeoff flange in which the hollow plunger is secured and which is located in the upper housing part,
a locking mechanism situated in the upper housing part,
a spring housing with the spring contained therein, which is rotatably mounted on the upper housing part by means of a rotary bearing,
a lower housing part which is fitted onto the spring housing in the axial direction.

The hollow plunger with valve body corresponds to a device disclosed in WO 97/12687. It projects partially into the cylinder of the pump housing and is axially movable within the cylinder. Reference is made in particular to FIGS. 1 to 4, especially FIG. 3, and the relevant parts of the description. The hollow plunger with valve body exerts a pressure of 5 to 60 Mpa (about 50 to 600 bar), preferably 10 to 60 Mpa (about 100 to 600 bar) on the fluid, the measured amount of active substance solution, at its high pressure end at the moment when the spring is actuated. Volumes of 10 to 50 microlitres are preferred, while volumes of 10 to 20 microlitres are particularly preferred and a volume of 15 microlitres per spray is most particularly preferred.

The valve body is preferably mounted at the end of the hollow plunger facing the valve body.

The nozzle in the nozzle body is preferably microstructured, i.e. produced by microtechnology. Microstructured nozzle bodies are disclosed for example in WO 94/07607; reference is hereby made to the contents of this specification, particularly FIG. 1 therein and the associated description.

The nozzle body consists for example of two sheets of glass and/or silicon firmly joined together, at least one of which has one or more microstructured channels which connect the nozzle inlet end to the nozzle outlet end. At the nozzle outlet end there is at least one round or non-round opening 2 to 10 microns deep and 5 to 15 microns wide, the depth preferably being 4.5 to 6.5 microns while the length is preferably 7 to 9 microns.

In the case of a plurality of nozzle openings, preferably two, the directions of spraying of the nozzles in the nozzle body may extend parallel to one another or may be inclined relative to one another in the direction of the nozzle opening. In a nozzle body with at least two nozzle openings at the outlet end the directions of spraying may be at an angle of 20° to 160° to one another, preferably 60° to 150°, most preferably 80° to 100°. The nozzle openings are preferably arranged at a spacing of 10 to 200 microns, more preferably at a spacing of 10 to 100 microns, most preferably 30 to 70 microns. Spacings of 50 microns are most preferred. The directions of spraying will therefore meet in the vicinity of the nozzle openings.

The liquid pharmaceutical preparation strikes the nozzle body with an entry pressure of up to 600 bar, preferably 200 to 300 bar, and is atomised into an inhalable aerosol through the nozzle openings. The preferred particle or droplet sizes of the aerosol are up to 20 microns, preferably 3 to 10 microns.

The locking mechanism contains a spring, preferably a cylindrical helical compression spring, as a store for the mechanical energy. The spring acts on the power takeoff flange as an actuating member the movement of which is determined by the position of a locking member. The travel of the power takeoff flange is precisely limited by an upper and lower stop. The spring is preferably biased, via a power step-up gear, e.g. a helical thrust gear, by an external torque which is produced when the upper housing part is rotated counter to the spring housing in the lower housing part. In this case, the upper housing part and the power takeoff flange have a single or multiple V-shaped gear.

The locking member with engaging locking surfaces is arranged in a ring around the power takeoff flange. It consists, for example, of a ring of plastic or metal which is inherently radially elastically deformable. The ring is arranged in a plane at right angles to the atomiser axis. After the biasing of the spring, the locking surfaces of the locking member move into the path of the power takeoff flange and prevent the spring from relaxing. The locking member is actuated by means of a button. The actuating button is connected or coupled to the locking member. In order to actuate the locking mechanism, the actuating button is moved parallel to the annular plane, preferably into the atomiser; this causes the deformable ring to deform in the annual plane. Details of the construction of the locking mechanism are given in WO 97/20590.

The lower housing part is pushed axially over the spring housing and covers the mounting, the drive of the spindle and the storage container for the fluid.

When the atomiser is actuated the upper housing part is rotated relative to the lower housing part, the lower housing part taking the spring housing with it. The spring is thereby compressed and biased by means of the helical thrust gear and the locking mechanism engages automatically. The angle of rotation is preferably a whole-number fraction of 360 degrees, e.g. 180 degrees. At the same time as the spring is biased, the power takeoff part in the upper housing part is moved along by a given distance, the hollow plunger is withdrawn inside the cylinder in the pump housing, as a result of which some of the fluid is sucked out of the storage container and into the high pressure chamber in front of the nozzle.

If desired, a number of exchangeable storage containers which contain the fluid to be atomised may be pushed into the atomiser one after another and used in succession. The storage container contains the aqueous aerosol preparation according to the invention.

The atomising process is initiated by pressing gently on the actuating button. As a result, the locking mechanism opens up the path for the power takeoff member. The biased spring pushes the plunger into the cylinder of the pump housing. The fluid leaves the nozzle of the atomiser in atomised form.

Further details of construction are disclosed in PCT Applications WO 97/12683 and WO 97/20590, to which reference is hereby made.

The components of the atomiser (nebuliser) are made of a material which is suitable for its purpose. The housing of the atomiser and, if its operation permits, other parts as well are preferably made of plastics, e.g. by injection moulding. For medicinal purposes, physiologically safe materials are used.

FIGS. 2a/b attached to this patent application, which are identical to FIGS. 6a/b of WO 97/12687, show the nebuliser (Respimat®) which can advantageously be used for inhaling the aqueous aerosol preparations according to the invention.

FIG. 2a shows a longitudinal section through the atomiser with the spring biased while FIG. 2b shows a longitudinal section through the atomiser with the spring relaxed.

The upper housing part (51) contains the pump housing (52) on the end of which is mounted the holder (53) for the atomiser nozzle. In the holder is the nozzle body (54) and a filter (55). The hollow plunger (57) fixed in the power takeoff flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomised. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

The nebuliser described above is suitable for nebulising the aerosol preparations according to the invention to produce an aerosol suitable for inhalation.

If the formulation according to the invention is nebulised using the method described above (Respimat®) the quantity delivered should correspond to a defined quantity with a tolerance of not more than 25%, preferably 20% of this amount in at least 97%, preferably at least 98% of all operations of the inhaler (spray actuations). Preferably, between 5 and 30 mg of formulation, most preferably between 5 and 20 mg of formulation are delivered as a defined mass on each actuation.

However, the formulation according to the invention may also be nebulised by means of inhalers other than those described above, e.g. jet stream inhalers.

Accordingly, in a further aspect, the invention relates to pharmaceutical formulations in the form of propellant-free inhalable solutions or suspensions as described above combined with a device suitable for administering these formulations, preferably in conjunction with the Respimat®. Preferably, the invention relates to propellant-free inhalable solutions or suspensions characterised by the combination of Compound 1 and Compound 2 according to the invention in conjunction with the device known by the name Respimat®. In addition, the present invention relates to the above-mentioned devices for inhalation, preferably the Respimat®, characterised in that they contain the propellant-free inhalable solutions or suspensions according to the invention as described hereinbefore.

The propellant-free inhalable solutions or suspensions according to the invention may take the form of concentrates or sterile inhalable solutions or suspensions ready for use, as well as the above-mentioned solutions and suspensions designed for use in a Respimat®. Formulations ready for use may be produced from the concentrates, for example, by the addition of isotonic saline solutions. Sterile formulations ready for use may be administered using energy-operated fixed or portable nebulisers which produce inhalable aerosols by means of ultrasound or compressed air by the Venturi principle or other principles.

Accordingly, in another aspect, the present invention relates to pharmaceutical compositions in the form of propellant-free inhalable solutions or suspensions as described hereinbefore which take the form of concentrates or sterile formulations ready for use, combined with a device suitable for administering these solutions, characterised in that the device is an energy-operated free-standing or portable nebuliser which produces inhalable aerosols by means of ultrasound or compressed air by the Venturi principle or other methods.

The Examples which follow serve to illustrate the present invention in more detail without restricting the scope of the invention to the following embodiments by way of example.

Preparation of Compound 1:

The synthesis of Compound 1, especially of Compound 1a and Compound 1b is described in the prior art, in particular in WO 02/30928, WO 03/000265 and in WO 02/32899.

The synthesis of Compound 2 is described e.g. in EP 0 771 794 A1, in particular as example 140 on page 109 therein.

Test on the Biological Activity

In diseases such as COPD and asthma neural pathways are believed to play an important role. These pathways can be classified under cholinergic pathways, in which acetylcholine is the end-transmitter, and tachykininergic pathways in which neuropeptides such as substance P or neurokinin A serve as end-transmitters. The example presented refers to the ability of these neural mechanisms to influence bronchospasm, but neural mechanisms can also be involved in regulation of mucus secretion or inflammatory processes, and the synergistic effects of Compound 1 and Compound 2 should not be considered to be restricted to effects on bronchospasm.

This example describes the effects of Compound 1 in combination with Compound 2 on the contraction of bronchus. As an example of Compound 1 tiotropium bromide was chosen, but this test may be performed with any Compound 1, in particular with Compound 1b, achieving comparable results. Male Hartley guinea pigs (300-700 g) were killed with CO₂ asphyxiation. The main bronchial rings were excised. The individual rings were opened by cutting through the cartilage and suspended vertically between parallel platinum wire field electrodes in the organ baths containing Krebs Henseleit (KH) solution (composition mmol/L: NaCl 119, KCl 4.7, MgSO₄ 1.2, CaCl₂ 2.5, KH₂PO₄ 1.2, NaHCO₃ 25 and glucose 11.7) at 37° C. and gassed with O₂/CO₂ (95:5, v/v). The preparations were allowed to equilibrate for 1 h with frequent washing. All experiments were performed in the presence of indomethacin (5 μmol/L) to prevent the formation of contractile prostaglandins. Contractions were measured by an isometric force transducer (TB-611T, Nihon Kohden, Tokyo, Japan) under a resting tension of 0.3 g. The preparation in this manner were contracted by electrical field stimulation (EFS: 30 V, 0.5 ms duration and a frequency of 8 Hz for 15 s). The EFS induced reproducible biphasic contraction. The fast phase contraction was mediated by acetylcholine (cholinergic phase). The slow phase contraction was mediated by tachykinins such as substance P and neurokinin A (tachykininergic phase) (Br. J. Pharmacol. 1996, 117 (5), 967-973). After the first EFS-induced contractions, the bronchi were treated with 10 nmol/L tiotropium bromide and/or 30 μmol/L Compound 2. The concentrations refer to the final bath concentrations. EFS-induced contractions were measured 20 min after the additions of the compounds Tiotropium bromide inhibited the cholinergic contraction. Compound 2 inhibited the tachykininergic contraction. The combination of tiotropium bromide and Compound 2 could inhibit both the cholinergic and tachykininergic contractions. The combination of the compounds inhibited the cholinergic contraction more strongly than tiotropium bromide itself. The combination of the compounds inhibited the tachykininergic contraction more potently than Compound 2 itself.

The inhibitions of both cholinergic response and tachykininergic response in the pulmonary system are effective in airway diseases such as asthma and COPD (Curr. Opin. Chem. Biol. 2000, 4 (4) 412-419, Eur. J. Pharmacol. 2001, 429 (1-3), 239-250). Therefore, the combination of active substances according to this invention is particularly suitable for treating inflammatory and/or obstructive diseases of the respiratory tract, particularly asthma or chronic obstructive pulmonary disease (COPD), and complications thereof such as pulmonary hypertension, as well as allergic and non-allergic rhinitis.

TABLE 1
Effects of compounds on the EFS-induced cholinergic and
tachykininergic contraction in the guinea pig main bronchus.
The EPS-induced contractions were measured in the absence
(Vehicle) or presence of 10 nmol/L tiotropium bromide
(Tiotropium bromide), 30 μmol/L Compound 2 (Compound 2) or
10 nmol/L tiotropium bromide and 30 μmol/l Compound 2
(Combination). Dimethyl sulfoxide (0.1%) and distilled water
(0.1%) were added as vehicles. n is the number of trials.
Table 1

% of the first contraction

| | | Cholinergic | Tachykininergic |
|---|---|---|---|
| Compounds | n | Contraction | contraction |
| Vehicle | 5 | 107.7 ± 2.8 | 98.7 ± 4.5 |
| Tiotropium | 5 | 59.7 ± 6.3 ** | 90.9 ± 8.3 |
| bromide | | | |
| Compound 2 | 5 | 105.8 ± 2.4 | 66.1 ± 6.5 ** |
| Combination | 5 | 43.2 ± 3.5 *** | 54.7 ± 10.3 ** |

Values are means ± S.E.M (standard error of the mean)
**, *** p < 0.01, 0.0001 vs the vehicle groups (Student's t-test).

### BRIEF DESCRIPTION OF THE DRAWINGS

FIG. 1 shows a particularly preferred inhaler for using the pharmaceutical combination.

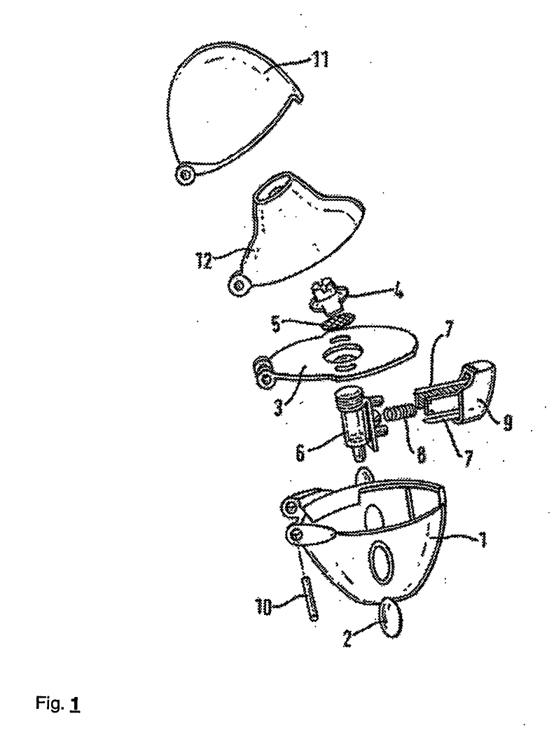

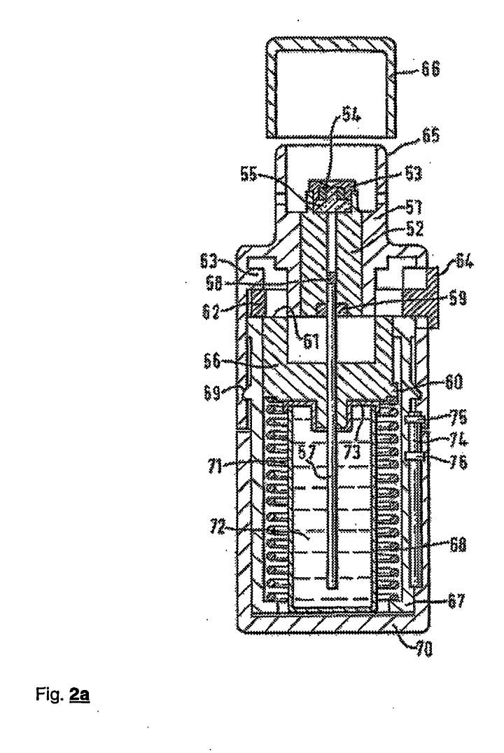

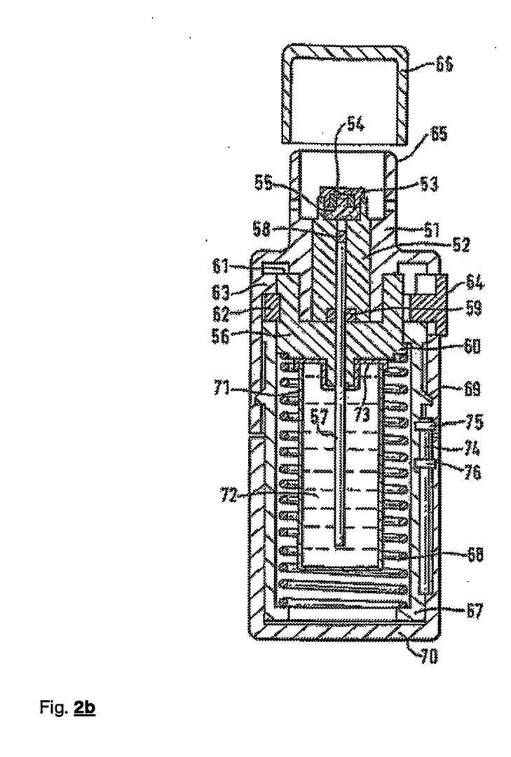

This inhaler (Handyhaler) for inhaling powdered pharmaceutical compositions from capsules is characterised by a housing 1 containing two windows 2, a deck 3 in which there are air inlet ports and which is provided with a screen 5 secured via a screen housing 4, an inhalation chamber 6 connected to the deck 3 on which there is a push button 8 provided with two sharpened pins 7 and movable counter to a spring 8, and a mouthpiece 12 which is connected to the housing 1, the deck 3 and a cover 11 via a spindle 10 to enable it to be flipped open or shut, as well as air holes 13 for adjusting the flow resistance.

FIG. 2a shows a longitudinal section through the atomiser with the spring biased.

FIG. 2b shows a longitudinal section through the atomiser with the spring relaxed.

The upper housing part (51) contains the pump housing (52) on the end of which is mounted the holder (53) for the atomiser nozzle. In the holder is the nozzle body (54) and a filter (55). The hollow plunger (57) fixed in the power takeoff-flange (56) of the locking mechanism projects partially into the cylinder of the pump housing. At its end the hollow plunger carries the valve body (58). The hollow plunger is sealed off by means of the seal (59). Inside the upper housing part is the stop (60) on which the power takeoff flange abuts when the spring is relaxed. On the power takeoff flange is the stop (61) on which the power takeoff flange abuts when the spring is biased. After the biasing of the spring the locking member (62) moves between the stop (61) and a support (63) in the upper housing part. The actuating button (64) is connected to the locking member. The upper housing part ends in the mouthpiece (65) and is sealed off by means of the protective cover (66) which can be placed thereon.

The spring housing (67) with compression spring (68) is rotatably mounted on the upper housing part by means of the snap-in lugs (69) and rotary bearing. The lower housing part (70) is pushed over the spring housing. Inside the spring housing is the exchangeable storage container (71) for the fluid (72) which is to be atomised. The storage container is sealed off by the stopper (73) through which the hollow plunger projects into the storage container and is immersed at its end in the fluid (supply of active substance solution).

The spindle (74) for the mechanical counter is mounted in the covering of the spring housing. At the end of the spindle facing the upper housing part is the drive pinion (75). The slider (76) sits on the spindle.

### EXAMPLES OF FORMULATIONS

A) Inhalable Powders:

TABLE 2

| Ingredients | μg per capsule |
|---|---|
| tiotropium bromide | 21.7 |
| Compound 2 | 250 |
| lactose | 4728.3 |
| total | 5000 |

TABLE 3

| Ingredients | μg per capsule |
|---|---|
| tiotropium bromide | 8.5 |
| Compound 2 | 500 |
| lactose | 4491.5 |
| total | 5000 |

TABLE 4

| Ingredients | μg per capsule |
|---|---|
| tiotropium bromide | 14.5 |
| Compound 2 | 900 |
| lactose | 4085.5 |
| total | 5000 |

TABLE 5

| Ingredients | μg per capsule |
|---|---|
| tiotropium bromide | 17.5 |
| Compound 2 | 1700 |
| lactose | 3282.5 |
| total | 5000 |

TABLE 6

| Ingredients | μg per capsule |
|---|---|
| tiotropium bromide | 24.5 |
| Compound 2 | 2300 |
| lactose | 2675.5 |
| total | 5000 |

TABLE 7

| Ingredients | μg per capsule |
|---|---|
| tiotropium bromide | 19 |
| Compound 2 | 3000 |
| lactose | 1981 |
| total | 5000 |

TABLE 8

| Ingredients | μg per capsule |
|---|---|
| tiotropium bromide | 20.5 |
| Compound 2 | 3750 |
| lactose | 2229.5 |
| total | 6000 |

TABLE 9

| Ingredients | μg per capsule |
|---|---|
| tiotropium bromide × H₂O | 22.5 |
| Compound 2 | 1150 |
| lactose | 3827.5 |
| total | 5000 |

TABLE 10

| Ingredients | μg per capsule |
|---|---|
| tiotropium bromide × H₂O | 19.0 |
| Compound 2 | 1350 |
| lactose | 3631 |
| total | 5000 |

TABLE 11

| Ingredients | μg per capsule |
|---|---|
| Compound 1b (as bromide) | 35 |
| Compound 2 | 900 |
| lactose | 4065 |
| Total | 5000 |

TABLE 12

| Ingredients | μg per capsule |
|---|---|
| Compound 1b (as bromide) | 75 |
| Compound 2 | 250 |
| lactose | 4675 |
| Total | 5000 |

TABLE 13

| Ingredients | μg per capsule |
|---|---|
| Compound 1b (as bromide) | 160 |
| Compound 2 | 650 |
| lactose | 4190 |
| Total | 5000 |

TABLE 14

| Ingredients | μg per capsule |
|---|---|
| Compound 1b (as bromide) | 265 |
| Compound 2 | 1350 |
| lactose | 3385 |
| Total | 5000 |

TABLE 15

| Ingredients | μg per capsule |
|---|---|
| Compound 1b (as bromide) | 375 |
| Compound 2 | 2350 |
| lactose | 2275 |
| Total | 5000 |

B) Propellant-Containing Aerosols for Inhalation:

1) Suspension Aerosol:

TABLE 16

| Ingredients | % by weight |
|---|---|
| tiotropium bromide | 0.003 |
| Compound 2 | 0.030 |
| soya lecithin | 0.2 |
| TG134a:TG227 = 2:3 | ad 100 |

2) Suspension Aerosol:

TABLE 17

| Ingredients | % by weight |
|---|---|
| tiotropium bromide | 0.0029 |
| Compound 2 | 0.040 |
| absolute ethanol | 0.5 |
| isopropyl myristate | 0.1 |
| TG 227 | ad 100 |

3) Suspension Aerosol:

TABLE 18

| Ingredients | % by weight |
|---|---|
| tiotropium bromide | 0.0029 |
| Compound 2 | 0.095 |
| absolute ethanol | 0.5 |
| isopropyl myristate | 0.1 |
| TG 227 | ad 100 |

4) Suspension Aerosol:

TABLE 19

| Ingredients | % by weight |
|---|---|
| tiotropium bromide | 0.029 |
| Compound 2 | 0.250 |
| absolute ethanol | 0.5 |
| isopropyl myristate | 0.1 |
| TG 227 | ad 100 |

TABLE 20

| Ingredients | % by weight |
|---|---|
| 1b (as bromide) | 0.020 |
| Compound 2 | 0.066 |
| soya lecithin | 0.2 |
| TG 134a:TG 227 = 2:3 | ad 100 |

TABLE 21

| Ingredients | % by weight |
|---|---|
| 1b (as bromide) | 0.0045 |
| Compound 2 | 0.0550 |
| absolute ethanol | 0.5 |
| isopropyl myristate | 0.1 |
| TG 227 | ad 100 |

1. A pharmaceutical composition, comprising
(a) an anticholinergic of the formula 1 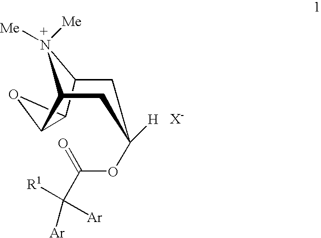
wherein
X⁻ represents a chlorine, bromine, iodine, methanesulphonate or trifluoromethanesulphonate anion;
R¹ represents hydroxy or methyl;
Ar represents phenyl or thienyl or a pharmacologically acceptable acid addition salt, solvate or hydrate thereof;
(b) a compound of the formula 2 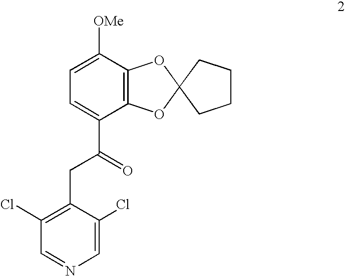
or a pharmacologically acceptable acid addition salt solvate, or hydrate thereof; and
(c) optionally, a pharmaceutically acceptable excipient.

2. The pharmaceutical composition according to claim 1, wherein the anticholinergic of the formula 1 is a compound of the formula 1a 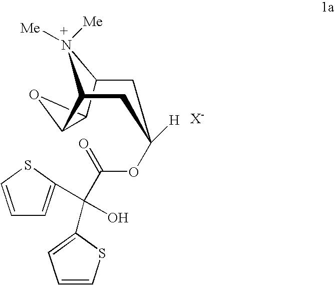
wherein
X⁻ represents a chlorine, bromine, iodine, methanesulphonate or trifluoromethanesulphonate anion.
or a pharmacologically acceptable acid addition salt, solvate, or hydrate thereof

3. The pharmaceutical composition according to claim 2, wherein X represents bromine.

4. The pharmaceutical composition according to claim 1, wherein the anticholinergic of the formula 1 is a compound of the formula 1b 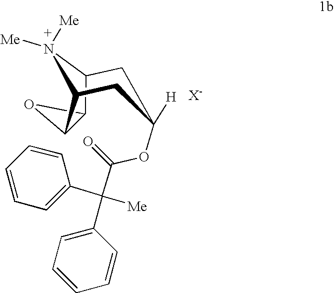
wherein
X⁻ represents a chlorine, bromine, iodine, methanesulphonate or trifluoromethanesulphonate anion.
or a pharmacologically acceptable acid addition salt, solvate, or hydrate thereof.

5. The pharmaceutical composition according to claim 4, characterised in that X represents bromine.

6. The pharmaceutical composition according to claim 1, wherein the anticholinergic of the formula 1 and the compound of the formula 2 are present either together in a single formulation or in two separate formulations.

7. The pharmaceutical composition according to claim 1, wherein the weight ratios of the anticholinergic of the formula 1 to the compound of the formula 2 are in the range from 1:4000 to 8:1.

8. The pharmaceutical composition according to claim 2, wherein the weight ratios of the compound of the formula 1a to the compound of the formula 2 are in the range from 1:4000 to 1:2.5.

9. The pharmaceutical composition according to claim 4, wherein the weight ratios of the compound of the formula 1b to the compound of the formula 2 are in the range from 1:4000 to 8:1.

10. The pharmaceutical composition according to claim 1, wherein the total dosage per single dose of the combination of the anticholinergic of the formula 1 and the compound of the formula 2 is from 25 to 10000 μg.

11. The pharmaceutical composition according to claim 1, wherein the composition is in the form of a formulation suitable for inhalation.

12. The pharmaceutical composition according to claim 11, wherein the composition is a formulation selected from among inhalable powders, propellant-containing metering aerosols and propellant-free inhalable solutions or suspensions.

13. The pharmaceutical composition according to claim 12, wherein the composition is an inhalable powder which contains the anticholinergic of the formula 1 and the compound of the formula 2 in admixture with physiologically acceptable excipients including monosaccharides, disaccharides, oligo- and polysaccharides, cyclodextrines, polyalcohols, salts, or mixtures thereof.

14. The Inhalable powder according to claim 13, wherein the excipient has a maximum average particle size of up to 250 μm.

15. The pharmaceutical composition according to claim 12, wherein the composition is an inhalable powder which contains only the anticholinergic of the formula 1 and the compound of the formula 2 as its ingredients.

16. The pharmaceutical composition according to claim 12, wherein the composition is a propellant-containing inhalable aerosol which contains the anticholinergic of the formula 1 and the compound of the formula 2 in dissolved or dispersed form.

17. The pharmaceutical composition in the form of a propellant-containing inhalable aerosol according to claim 16, wherein the propellant is a, hydrocarbons or a halohydrocarbon.

18. The pharmaceutical composition in the form of a propellant-containing inhalable aerosol according to claim 17, wherein the propellant gas is TG11, TG12, TG134a (1,1,1,2-tetrafluoroethane), TG227 (1,1,1,2,3,3,3-heptafluoropropane) or a mixture thereof.

19. The pharmaceutical composition according to claim 12, wherein the composition is a propellant-free inhalable solution or suspension which contains water, ethanol or a mixture of water and ethanol as solvent.

20. The pharmaceutical composition in the form of an inhalable solution or suspension according to claim 19, wherein the pH is 2 to 5.

21. A capsule comprising an inhalable powder according to claim 13.

22-24. (canceled)

25. A method of prophylaxis of, treating of, or reducing the exacerbations associated with pulmonary diseases comprising administering to a patient in need thereof an effective amount of a pharmaceutical composition according to claim 1 either in a single combined form, separately, or separately and sequentially where the sequential administration is close in time, or remote in time.

26. The method according to claim 25 wherein the pulmonary disease is asthma, COPD, or another obstructive airways disease exacerbated by bronchial hyperreactivity and bronchospasm.

27. The method according to claim 25 wherein said administration by inhalation comprises simultaneous or sequential delivery of said combination of therapeutic agents, comprising the anticholinergic of the formula 1 and the compound of the formula 2, in the form of an aerosol or dry powder dispersion.

28. The method according to claim 25, wherein the anticholinergic of the formula 1 is the compound of the formula 1a.

29. The method according to claim 25, wherein the anticholinergic of the formula 1 is the compound of the formula 1b.

30. A package comprising a pharmaceutical composition according to claim 1 for insertion into a device of simultaneous or sequential delivery of said pharmaceutical composition in the form of an aerosol or dry powder dispersion, to a mammal in need of thereof.

31. An Inhaler comprising a pharmaceutical composition according to claim 1 for simultaneous or sequential delivery of said pharmaceutical composition in the form of an aerosol or dry powder dispersion, to a mammal in need thereof.

32. A Pharmaceutical composition, comprising an anticholinergic in combination with the compound of the formula 2 optionally in the form of a pharmacologically acceptable acid addition salt thereof, optionally in the form of a solvate or hydrate and optionally together with a pharmaceutically acceptable excipient.

33-37. (canceled)
